(19) United States
(12) Patent Application Publication
(10) Pub. No.: US 2026/0102319 A1
(43) Pub. Date: Apr. 16, 2026

(54) PHOTORESPONSIVE AZOBENZENE FUNCTIONAL NANOGELS AND THEIR USE IN DENTAL ADHESIVES

(71) Applicant: The Regents of the University of Colorado, a Body Corporate, Denver, CO (US)

(72) Inventors: Devatha P. Nair, Denver, CO (US); Dixa Gautam, Aurora, CO (US); Rinku Trevedi, Englewood, CO (US); Jeffrey Stansbury, Centennial, CO (US)

(21) Appl. No.: 19/117,295

(22) PCT Filed: Oct. 12, 2023
(86) PCT No.: PCT/US2023/076642

§ 371 Date: Mar. 31, 2025

Related U.S. Application Data
(60) Provisional application No. 63/416,144, filed on Oct. 14, 2022.

(51) Int. Cl. A61K 6/62; A61K 6/30

(52) U.S. Cl. CPC A61K 6/62; A61K 6/30

(57) ABSTRACT

A light responsive azobenzene functional nanogel comprises the solution polymerization product of an azobenzene having a first reactive group and a monomer mixture comprising one or more polymerizable monomers having a second reactive group reactive with the first reactive group. The light responsive azobenzene can be combined with a functional material and solvent to form a photomotive composition characterized in that irradiating with light induces movement of the composition. The photomotive composition can be a dental adhesive as a single component system or in the form of a kit.

### FIELD OF THE INVENTION

The field of the invention is particularly photoresponsive (particularly photomotive) compositions, and their use as dental adhesives.

### BACKGROUND OF THE INVENTION

The management and replacement of failed restorations account for about fifty percent of restorative work done in dentistry and amounts to $5 billion in expenditures annually in the USA alone. The preference for composites is a global trend, and these aesthetically superior tooth-colored restorations continue to replace amalgam restorations, with over eight hundred million composite restorations placed worldwide in 2015. However, an average composite lasts only five to seven years. The adhesive interface, which connects the composite to the underlying tooth structure, has been identified as a weak link in the restoration and a reason composites fail. Bonding with most of the adhesive systems available in the market today usually results in only partially infiltrated collagen fibrils, thus favoring degradation and failure of the composite. In addition to the loss of function, the failure of the restoration at the adhesive interface can be responsible for dentin hypersensitivity, the susceptibility to secondary caries, and marginal staining, all factors that contribute to the growing clinical burden associated with failed restorations and patient discomfort/dissatisfaction. For the over sixty-three million Americans who lack access to dental insurance coverage, the thousands of people who inhabit the so-called ‘dental deserts’ of the U.S. and the underserved special needs populations, the frequent replacement, and removal of failed restorations can be daunting for financial, geographical, and behavioral reasons.

A long-term, stable bond with dentin is desirable for the success of restorations. To achieve this in composite restorations, adhesive resin penetration and collagen encapsulation within the decalcified dentin are useful for both the protection of the exposed collagen layer from external insults over time and the ability to strongly anchor the composite to tooth via the hybrid layer. Currently, during adhesive placement, the expectation is that the resins will flow into the demineralized dentin surface via simple diffusion until the depth of etching, homogenously encapsulate the exposed collagen within the hybrid layer, and micromechanically bond the adhesive layer to the tooth. However, non-homogeneous resin penetration often leaves water-rich regions with exposed collagen and available cleavage sites. Upon polymerization, this makes the adhesive-tooth interface vulnerable to mechanical stresses and degradation via matrix metalloproteinases (MMPs) and cathepsins over time. Relying solely on micromechanical bonds to anchor the restoration to the tooth also leaves the adhesive-tooth interface susceptible to surface strains (delivered via masticatory forces and/or temperature), which can further weaken and bend/distort tooth structures, leading to gap formation and marginal leakage around restorations.

Therefore, a need remains for improved dental adhesives.

### SUMMARY OF THE INVENTION

Disclosed herein is a light responsive azobenzene nanogel comprising the reaction product (e.g., solution polymerization product) of an azobenzene having a first reactive group and a monomer mixture comprising one or more polymerizable monomers having a second reactive group reactive with the first reactive group.

Also, disclosed herein is a composition (e.g., a photoresponsive or photomotive composition) comprising such a light responsive azobenzene functional nanogel, a polymer or a polymer precursor, and a solvent, characterized in that when the composition is exposed to light the light responsive azobenzene nanogel facilitates movement of the polymer or the polymer precursor. The composition can be a dental adhesive when an azobenzene functional nanogel is combined with a precursors used as dental adhesives.

In addition, disclosed herein is a kit comprising a first container having contents comprising a free radical initiator and at least one of (a) the light responsive azobenzene functional nanogel as described herein and (b) a polymer or a polymer precursor; and a second container having contents comprising a catalyst and at least one of (a) the light responsive azobenzene functional nanogel as described herein and (b) a polymer or a polymer precursor.

Further, disclosed herein is a method comprising providing a substrate, applying a to the substrate where a composition comprising a photoresponsive azobenzene functional nanogel as described herein, a functional component and a solvent, and irradiating the composition on the substrate with light to induce movement of the composition on the substrate to a desired location on the substrate.

### BRIEF DESCRIPTION OF THE DRAWINGS

Referring now to the figures, which are exemplary embodiments, and wherein the like elements are numbered alike.

FIG. 1 are confocal images showing diffusion of dental adhesive into etched dentinal tubules with and without the azobenzene functional nanogel.

FIG. 2 are photographs showing response of a control composition and a composition including an azobenzene functional nanogel to exposure to light.

FIG. 3 is a schematic of an example of an azobenzene functional nanogel as disclosed herein which further includes unreacted vinyl groups.

FIG. 4 is a UV-visible light spectroscopy of azobenzene functional nanogels as disclosed herein.

FIG. 5 is a schematic of a portion of a surface of azobenzene functional nanogels as disclosed herein having various pendant groups.

FIG. 6 is a schematic of a method as disclosed herein.

FIG. 7 is a graph of flexural strength test results for a composition comprising a dental adhesive with azobenzene-functional nanogel having carboxylic acid functionality.

FIG. 8 is a flow chart of an example of a method of making and using a photomotive composition as described herein.

### DETAILED DESCRIPTION OF THE INVENTION

Disclosed herein are azobenzene functional nanogels. These azobenzene functional nanogels are light responsive and can be added to compositions (such as dental compositions) to enable direction controlled movement of such composition based on irradiation with an appropriate wavelength of light. The light can be visible light. The light can have a wavelength of 380 to 750, or 430 to 480 nanometers (nm). The photoresponsive compositions generally will move away from the light upon exposure to such light. The inclusion of the azobenzene functionality in the nanogel enables use of the benefit of the photoisomerism of azobenzene from the hydrophobic trans-isomeric form to the hydrophilic cis-isomeric form in biomedical applications. In other words the nanogel structure can compatibilize the azobenzene to enable its use in polar solvents (e.g., water and alcohol). Such use was previously precluded by the extreme hydrophobicity and insolubility of azobenzene molecules.

### The Azobenzene Functional Nanogel

The azobenzene functional nanogel can be formed by solution polymerization of an azobenzene having a reactive group with monomers desired to form the nanogel. General methods of forming nanogels are disclosed, for example, in WO2013/142354 and U.S. Pat. No. 9,133,838. For example, the polymerization can occur via reaction of a hydroxyl group with reaction of an isocyanate group using a catalyst, such as dibutyltin dilaurate or an amine catalyst. As a more specific example, a hydroxyl functional azobenzene can be reacted with monomers having functional groups that react with hydroxyl groups to form the azobenzene functional nanogel.

As another example, an azobenzene having ethylenic unsaturation (e.g., acrylate or methacrylate functionality) can be reacted in solution polymerization via free radical polymerization with other monomers having ethylenic unsaturation to form the nanogel. In this case a free radical initiator, such as a peroxide can be used.

The solvent for the solution polymerization can be, for example, a ketone such as methyl ethyl ketone, an aromatic solvent such as toluene, an alcohol such as ethanol or the like.

The azobenzene functionalized nanogel acts as a compatibilizing agent between the material of the tooth and a photoactive material that is used to fill cavities in the tooth.

For example, the azobenzene could have one of the following structures

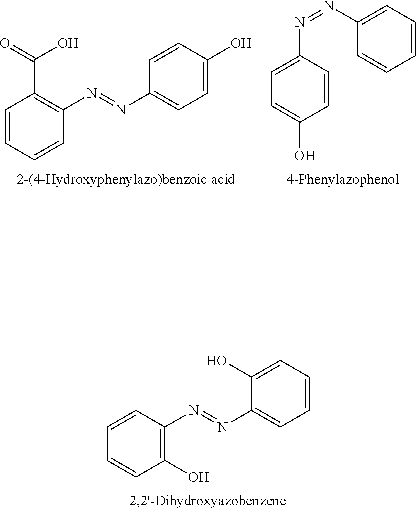

The other monomers (that have functional groups that can react to form the azobenzene functional nanogel) can comprise polyols, monoisocyanates, polyisocyanates (e.g., diisocyantate), ethylenically unsaturated monomers, such as acrylates and methacrylates (including diacrylates or dimethacrylates), cyanoacrylates, carboxylic acid functional monomers and the like. Note that the term (meth)acrylate or (meth)acrylic is used herein to denote acrylates and/or methacrylates and acrylic and/or methacrylic acids, respectively. In an embodiment, methacrylates and methacrylic materials are preferred. Having at least one monomer that has a functionality greater than 2, such as, for example, 3 or greater, 4 or greater, enables formation of cross-linking in the nanogel. Specific examples of monomers include the following:

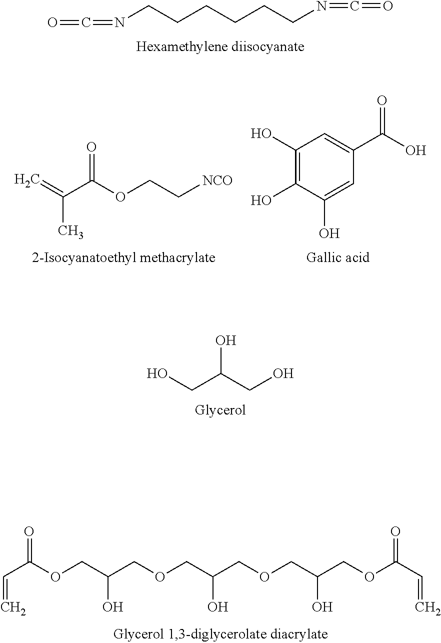

Additional monomers can include divinyl monomer such as one or more of ethylene glycol di(meth)acrylate, tetraethyleneglycoldi(meth)acrylate (TTEGDMA), urethane dimethacrylate (UDMA), the condensation product of bisphenol A and glycidyl (meth)acrylate, 2,2-bis[4-(3-methacryloxy-2-hydroxy propoxy)-phenyl]propane (bis-GMA), ethoxylated bisphenol-A-di(meth)acrylate (BisEMA), hexanediol di(meth)acrylate, polyethylene glycol dimethacrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl (meth)acrylate, divinyl benzene, bis(meth)acrylamide, or 1,3-diglycerolatediacrylate, or monovinyl monomers such as styrene and derivatives thereof (styrenics), vinyl acetate, maleic anhydride, itaconic acid, N-alkyl (aryl) maleimides and N-vinyl pyrrolidone, vinyl pyridine, acrylamide, methacrylamide, N,N-dialkylmethacrylamides or acrylonitrile, preferably polyethoxyethyl methacrylate.

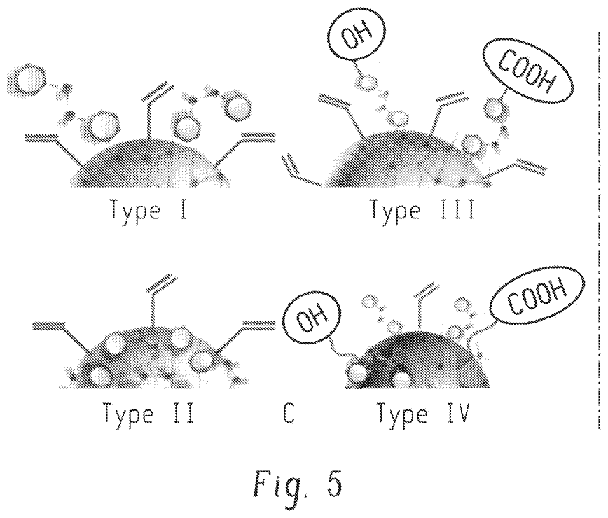

Selection of the monomers can enable formation of nanogels that can have vinyl and azobenzene functionality (Type 1 as in FIG. 5); vinyl, azobenzene and hydroxyl functionality (Type II as in FIG. 5), or azobenzene, vinyl, hydroxyl and carboxylic acid functionality (Types III and IV in FIG. 5). The additional functional groups can enhance reactivity or other properties of the nanogel. For example, hydroxyl and/or carboxylic acid functionality on the azobenzene functional nanogel in a dental adhesive can induce intermolecular and intermicrofibrillar crosslinks, along with hydrophobic interactions that can enhance the mechanical properties and fibril biostability, which in turn decreases their susceptibility to bacterial collagenase and mahrix metalloproteinases (MMPs).

The amounts of monomers used in synthesis of the azobenzene functional nanogels can be varied to adjust various features of the nanogel. For example, a weight ratio of hydroxyl functional azobenzene to polyol (e.g., glycerol or gallic acid) can be 20:80 to 90:10, or 30:80 to 85:15, or 40:60 to 80:20, or 50:50 to 75:25. The hydroxyl functional azobenzene and polyol can be reacted with a stoichiometric amount of diisocyanate and isocyanoalkyl (meth)acrylate. The weight ratio of the diisocyanate:isocyanoalkyl (meth) acrylate can be 20:80 to 90:10, or 30:80 to 85:15, or 40:60 to 80:20, or 50:50 to 75:25.

The azobenzene functional nanogels can be in the form of a particulate. The particulate can swell when in a suitable solvent or collapse upon removal of solvent. The particulate can have a diameter in the range of 1 to 60 nm, preferably 2 to 50 nm and more preferably 5 to 40 nm. The azobenzene functional nanogels can, for example, have weight average molecular weights in the range of 10,000 to 50,000 Daltons (Da) as measured by Gel Permeation Chromatography.

The azobenzene functional nanogels as disclosed herein can swell and disperse in solvated networks.

Photomotive Compositions and their Use

The addition of the azobenzene functional nanogels to a composition comprising a functional material can assist in directing the functional material to the location where it is desired. Specifically by irradiating the composition with light, the composition will move in a direction away from the light.

The photoresponsive compositions can thus comprise the azobenzene functional nanogel, a functional material, such as a polymer or polymer precursor composition and a solvent. These composition can be applied to a substrate, and irradiated with light to direct the composition in a direction away from the light.

For example, the photoresponsive composition could comprise as the functional component a dental adhesive. The dental adhesive can be a commercially available dental adhesive. The dental adhesive can comprise divinyl monomer, preferably a di(meth)acrylate and a monovinyl monomer, preferably a monacrylate. Examples of divinyl monomers include ethylene glycol di(meth)acrylate, hexanediol di(meth)acrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl(meth)acrylate, urethane di(meth)acrylate, 2,2′-bis[4-(3-methacryloxy-2-hydroxy propoxy)-phenyl]propane (bis-GMA), ethoxylated bisphenol-A-di(meth)acrylate, divinyl benzene, or combinations of two or more thereof. Examples of monovinyl monomers include C1-C20 alkyl(meth)acrylate, such as ethyl(meth)acrylate or isobornyl(meth)acrylate; an aromatic (meth)acrylate, such as 2-phenoxyethyl(meth)acrylate, benzoyl(meth)acrylate, or phenyl(meth)acrylate; (meth)acrylic acid, and combinations of two or more thereof. As a more specific example, the adhesive can include bis-GMA and hydroxyethyl methacrylate (HEMA).

The azobenzene functional nanogel can be used in the photoresponsive composition in amounts of, for example, at least 0.2, at least 0.5 or at least 1 weight % up to 5, up to 4, up to 3 or up to 2.5 weight % based on total weight of the photoresponsive composition.

The solvent can be, for example, water, alcohol, or a combination thereof. For example, the solvent can comprise, consist essentially or consist of ethanol.

The photoresponsive composition can further include an initiator such as a photopolymerization initiator or a free radical initiator. Examples of photopolymerization initiators include camphorquinone/amine, trimethylbenzoyldiphenyl phosphine oxide (TPO), 2,2-dimethoxy-2-phenyl acetophenone (DMPA). Examples of free radical initiators includes peroxides such as benzoyl peroxide.

The photoresponsive composition can further include a catalyst, such as N, N-dimethyl-p-toluidine, particularly when a free radical initiator is used.

The photoresponsive composition can be provided in a single component form. Alternatively, the photoresponsive composition can be provided in a kit form. The kit can comprise a first container comprising a free radical initiator; and a second container comprising a catalyst, provided the azobenzene functional nanogel and the polymer or polymer precursor are included in at least one of the containers. Both containers can contain the azobenzene functional nanogel and the polymer or polymer precursor. The kit can provide a mechanism for mixing the contents of the two containers. For example, the containers can be cartridges that combine and mix the contents by injection through a single orifice.

The photoresponsive composition can be applied to any substrate where it is desirable to apply the functional component. Particularly, the substrate can include small pores, fissures or the like which can make application to all the exposed surfaces challenging. By applying the photoresponsive composition and then irradiating with light the composition can be driven into such pores or fissures better insuring good and complete coverage of exposed surfaces of the substrate.

The photoresponsive composition can have a viscosity of less than 40 mPa-s as measured on a Brookfield viscometer.

For example, the substrate can be dentin, and particularly dentin that has been conditioned (or etched) in preparation for application of a dental adhesive or dental composition.

The flow chart in FIG. 8 describes an exemplary method from synthesis of the azobenzene nanogel through use of a photomotive composition. The functionalized material can be, for example, a dental adhesive composition. The substrate can be, for example, a tooth or dentin conditioned in advance of applying the photoresponsive material. The conditioned substrate can include pores or fissures and the irradiation with light can cause the photoresponsive material to diffuse into those pores or fissures. If a photoinitiator is included in the functional material the irradiation can also initiate cure of the photoresponsive material.

### EXAMPLES

### Example 1—Synthesis of Azobenzene Functional Nanogel and Photoresponsive Composition Comprising the Same

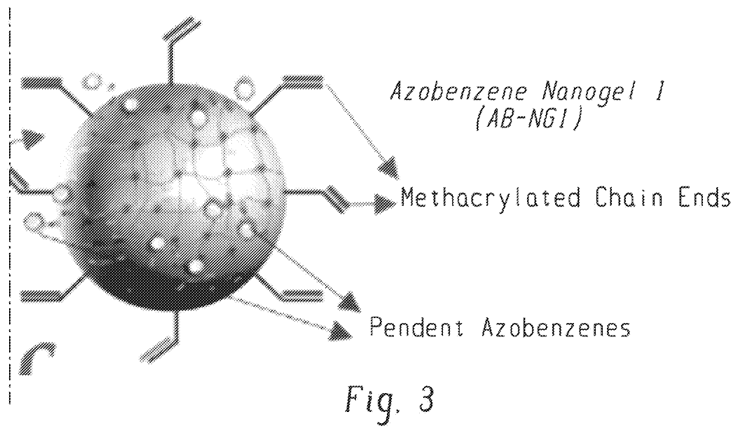

Photodynamic nanogel with azobenzene (AB-NG1) was synthesized via a stoichiometric step-growth solution polymerization reaction in which 70-30 wt. % of 4-hydroxyazobenzene and glycerol is reacted with a stoichiometric of the ratio of hexamethylene diisocyanate and isocyantoethyl methacrylate (also in 70-30 ratio) with dibutyltin dilaurate as catalyst in four times the amount of toluene under ambient conditions for twelve hours. Mid-FTIR (Fourier transform infrared spectroscopy) peaks for the isocyanate and alcohol were monitored until the hydroxyl and isocyanate peaks were consumed (i.e., 100% conversion). The crude reaction mixture was purified by precipitation in hexanes (10-fold excess) to obtain AB-NG1. The AB-NG1 was characterized with triple-detector GPC in tetrahydrofuran (M_{w}=12 kiloDaltons (kDa) and Rₕ=1.74 nm). FIG. 3 is a conceptual schematic of AB-NG1.

### Example 2

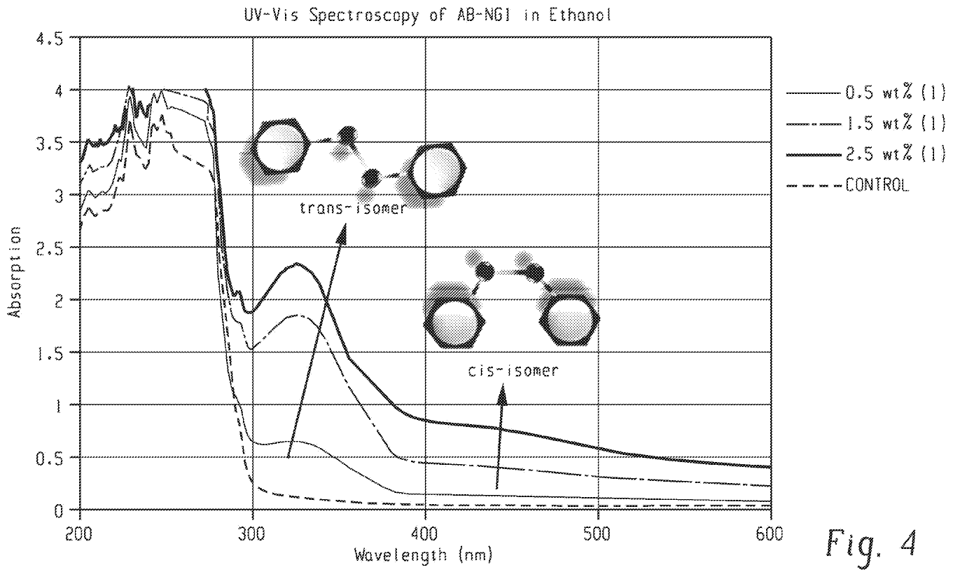

UV-Vis Spectroscopy of AB-NG1 in ethanol is shown in FIG. 4. Characteristically, the AB trans-isomer absorbs at wavelengths of about 300-400 nm while the cis isomer absorbs at wavelengths of about 400-500 nm. The presence of one continuous peak indicates AB present in both cis and trans isomers at the resting state in ethanol, (see arrows in FIG. 4) via UV-Vis Spectroscopy. This photostationary state, in which the transient, hydrophilic cis state is also stabilized via ethanol makes AB an ideal candidate to undergo the cyclical trans-cis-trans isomerization when illuminated.

### Example 3

The AB-NG1 was combined with a 60/40 wt/wt mixture of Bis-GMA/HEMA and 12 weight % ethanol (referred to as B/H/E). The viscosity was measured using parallel plate viscometer and compared to a B/H/E control during irradiation. See Table 1. Up to 2.5 wt. % addition of AB-NG1 to the B/H/E mix (10 microliters (μL)) did not significantly alter the viscosity (measured by parallel plate viscometer) compared with the B/H/E control during irradiation from the Elipar™ dental lamp (430-480 nm, 700 milliwatts per square centimeter (mW/cm²), 60 second (s) irradiation, no initiator). The nanogel (NG) swell within favorable solvents and monomers, and therefore, the inter-particular distance between NG within formulations can be very low. Hence, the viscosity will increase sharply with NG concentration once the percolation threshold—whereupon NG start overlapping with each other—is reached. At 5 wt. %, the viscosity was 39.3±6.4 milliPascal-seconds (mPa-s) while at 10 wt. % it had risen sharply to 91±3 mPa-s indicating that percolation threshold for this network lay between 5 and 10 wt. % AB-NG1.

Due to absorbance in the visible region, AB-NG1 can function as a photoswitchable agent to enhance resin mobility while also modestly inhibiting the onset of adhesive polymerization, which creates a window of opportunity for resin translocation followed by fixing the resin in place by polymerization. The photoinitiators camphorquinone and ethyl 4-N,N′-dimethylaminobenzoate (CQ-EDMAB, 1:1 weight ratio, total 2 wt. %), required about 4 minutes of light exposure (Elipar™ 430-480 nm at 700 mW/cm²) for >95% double-bond conversion of the resin (quantified via Near-FTIR spectroscopy by measuring the methacrylate peak area before and after polymerization at 6163 per centimeter (cm⁻¹)). However, the incorporation of an additional redox initiating system benzoyl peroxide and N, N-dimethyl-p-toluidine (BPO-DMT, 2:1 wt:wt, 2 wt. %) resulted in >95% double-bond conversion via two, 20 second exposures (Table 1). Fine-tuning resin mobility with polymerization kinetics and double-bond conversion on clinically relevant timescales is a key component of this study and will be characterized via adjustment of the AB-NG concentration and irradiation conditions.

To study surface properties of polymerized substrates, 15 μL of water was placed on polymerized disc specimens of AB-NG1/B/H/E (t=1 mm, d=5 mm), and the angle at the interface was measured for each sample on a goniometer (Table 1). With a CA of 53°, the hydrophobicity of the AB-NG1 dominated the AB-NG1/B/H/E networks, indicating that upon polymerization, the hydrophobic trans state of AB dominate, which is also its stable resting state.

TABLE 1

| | | Double-bond Conversion (%) | | |
|---|---|---|---|---|
| | | | Dual-initiators | Contact |
| | Viscosity | Photoinitiator | (photo + redox) | Angle |
| Formulation | (mPa s) | (4 min) | (40 s) | (°) |
| B/H/E | 32.1 ± 5.0 | 97.3 ± 1.06 | 98.6 ± 0.3 | 28 ± 1 |
| AB-NG1/B/H/E | 37.0 ± 8.0 | 97.6 ± 0.6 | 98.2 ± 0.5 | 53 ± 2 |

### Example 4

The cytocompatibility of the synthesized AB-NG1 within B/H/E networks was evaluated using an elution MTT (3-(4,5-dimethylthiazol-2-yl)-2,5-diphenyltetrazolium bromide) assay following a 24 hour incubation period. Briefly, the AB-NG1/B/H/E networks polymerized into discs (d=10 millimeter (mm), t=0.9 mm, n=3) and placed in cell media for 24 hours @37° C. to uptake extractables. The extract media was diluted to different concentrations (100, 50, 25, 12.5, 6.25, 0%) and added to L929 mouse fibroblast cells (ECACC 85011425) and incubated for an additional 24 hours after which 0.5 milligrams per milliliter (mg/mL) of 3-(4,5-dimethylthiazol-2-yl)-2,5-diphenyltetrazolium bromide (MTT reagent) was added to the wells. The percent viability was calculated using the formula: % Cell Viability=(A_{Experimental group}/A_{control group})*100. The highest concentration of AB-NG1/B/H/E evaluated at 5 wt. % showed approx. 100% cell viability.

### Example 5

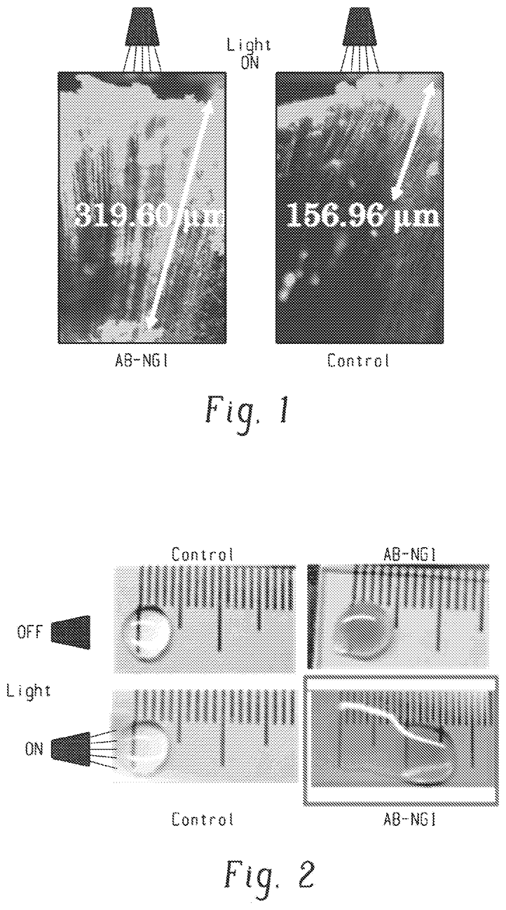

The effect of the inclusion of AB-NG1 at 1% by weight in B/H/E can be seen in FIGS. 1 and 2. In FIG. 1 it is shown that after applying such composition to etched dentin and applying light at 430 to 480 nm wavelength there is substantially more infiltration of dentin tubules from the AB-NG1/B/H/E composition that there is for the control that comprised only the B/H/E. FIG. 1 is confocal laser canning microscopy. Confocal laser scanning microscopy is a non-destructive, high-resolution method to screen formulations and quantify their ability to penetrate the demineralized dentin and dentinal tubules and form homogenous networks. An untethered dye (here Rhodamine was used) is added at 0.1 weight percent for imaging. In FIG. 2 droplets of the control and the AB-NG1/B/H/E were applied to a substrate and irradiated while being held in a horizontal position. As shown, the AB-NG1/B/H/E composition moved away from the light while the control did not move.

### Example 6

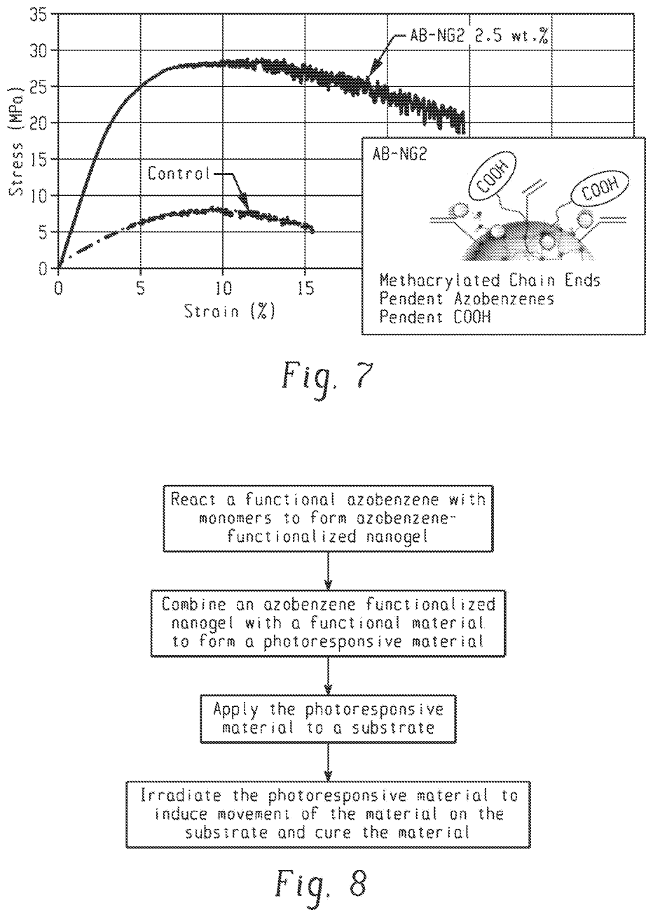

A nanogel was synthesized substantially as set out in Example 1 except the glycerol was replaced with gallic acid to provide carboxylic acid functionality to form AB-NG2. AB-NG2 at 2.5 wt. % was incorporated within 60:40 UDMA/HEMA wt/wt mixture with 12 wt. % ethanol. Bar specimens with dimensions of 25 millimeter (mm)×2 mm×2 mm, light-cured between glass slides in an elastomer mold by exposure to Elipar™ at 700 milliwats per square centimeter (mW/cm²) for 2 minutes on each side were prepared for flexural tests Flexural tests (n=8) show a significant increase in modulus and toughness with the addition of the AB-NG2 (Modulus=336±14 MegaPascals (MPa), Toughness=5±1 milliJoules per cubic millimeter (mJ/mm³)) in comparison with a control (Modulus 151±8 MPa, Toughness=0.8±0.2 mJ/mm³). (FIG. 7). This indicates that providing phenolic and non-phenolic COOH and OH functionality can enhance the strength of the hybrid layer via intermolecular and intermicrofibrillar bonding with dentin.

### Example 7—Prophetic

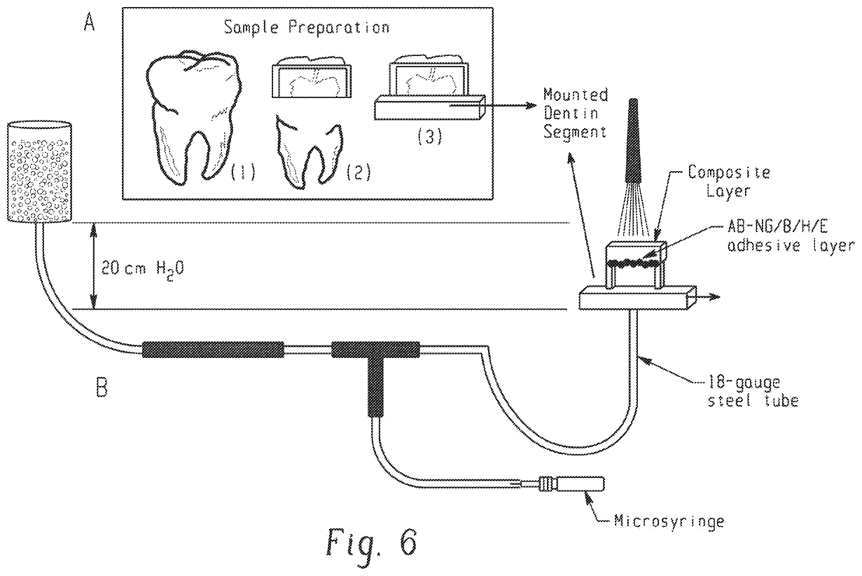

It would be desirable to have dental bonding agents that counter pulpal pressure and the resulting water barrier that resists the penetration of the adhesive resin during bonding. The light-assisted diffusion is tested to drive resins up to the etch depth created via the etching agent, and not disproportionately displace the water present within the dentinal tubules and/or run the risk of collapsing the hydrated collagen mesh. The driving force of the AB-NG/adhesive formulations is tailored to match the pulpal pressure up to the etch depth and the ability of the AB-NG adhesive formulation to infiltrate the collagen fibers up to the depth of conditioning is evaluated, in the presence of simulated pulpal pressure. By providing a counterforce that keeps excessive water from the pulpal pressure at bay, we hypothesize that the light-propelled movement of the adhesive monomers will provide a stable interface with enhanced strength, which can be quantified via an increase in p TBS of the AB-NG samples. The average value of pulpal pressure in vital teeth is 20 centimeters (cm) H₂O. A test set-up is illustrated by FIG. 6.

The mounted segments will be placed in the experimental set-up and exposed to simulated pupal pressures while the samples are subjected to acid etching with 37% phosphoric acid (Scotchbond Universal Etchant, 3M ESPE) for 15 seconds. Under constant pulpal pressure, the adhesive layer will be placed and irradiated twice for 20 seconds after which composite buildups will be constructed (2 mm in height, 2 layers) using Filtek Z-100 (3M ESPE) as shown in Scheme 3B. Samples will be light-cured using Elipar™ at 700 mW/cm².

This disclosure further encompasses the following aspects.

Aspect 1: A light responsive azobenzene functional nanogel comprising the reaction product of an azobenzene having a first reactive group and a monomer mixture comprising one or more polymerizable monomers having a second reactive group reactive with the first reactive group.

Aspect 2: The light responsive azobenzene functional nanogel of Aspect 1 wherein the monomer mixture comprises one or more monovinyl monomers and one or more divinyl monomers and a difunctional chain transfer agent.

Aspect 3: The light responsive azobenzene functional nanogel of any of the preceding Aspects wherein the first reactive group of the azobenzene is an ethylenically unsaturated group, a hydroxyl group, or an isocyanate group.

Aspect 4: The light responsive azobenzene functional nanogel of Aspect 2 or 3 wherein divinyl monomer comprises one or more of ethylene glycol di(meth)acrylate, tetraethyleneglycoldi(meth)acrylate (TTEGDMA), urethane dimethacrylate (UDMA), the condensation product of bisphenol A and glycidyl (meth)acrylate, 2,2-bis[4-(3-methacryloxy-2-hydroxy propoxy)-phenyl]propane (bis-GMA), ethoxylated bisphenol-A-di(meth)acrylate (BisEMA), hexanediol di(meth)acrylate, polyethylene glycol dimethacrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl (meth)acrylate, divinyl benzene, bis(meth)acrylamide, or 1,3-diglycerolatediacrylate.

Aspect 5: The light responsive azobenzene functional nanogel of any one of Aspects 2 to 4 wherein the monovinyl monomer comprises (meth)acrylates and acrylates, styrene and derivatives thereof (styrenics), vinyl acetate, maleic anhydride, itaconic acid, N-alkyl (aryl) maleimides and N-vinyl pyrrolidone, vinyl pyridine, acrylamide, methacrylamide, N,N-dialkylmethacrylamides or acrylonitrile, preferably polyethoxy ethyl methacrylate.

Aspect 6: The light responsive azobenzene functional nano gel of any one of Aspects 2-5 wherein the difunctional chain transfer agent comprises mercaptoethanol, mercaptopropanol, 3-mercapto-2-butanol, 2-mercapto-3-butanol, 3-mercapto-2-methyl-butan-1-ol, 3-mercapto-3-methyl-hexan-1-ol-3-mercaptohexanol, 3-mercaptopropionic acid, or cysteine.

Aspect 7: The light responsive azobenzene functional nanogel of Aspect 1 or 2 wherein mixture comprises comprising an azobenzene comprises a hydroxyl functional group or an isocyanate functional group and the monomer mixture comprises a polyol, a cyanoalkyl (meth)acrylate, and a diisocyanate.

Aspect 8: The light responsive azobenzene functional nanogel of Aspect 7 wherein the polyol is a glycerol, the cyanoalkyl (meth)acrylate is isocantoethyl methacrylate, and the diisocyanate is hexamethylene diisocyanate.

Aspect 9: The light responsive azobenzene functional nanogel of any one of Aspects 1-8 which is spherically cross-linked and swells in a solvent selected from water, alcohol or combinations there.

Aspect 10: The light responsive azobenzene functional nanogel of any one of Aspects 1-9 further comprising one or more of unreacted vinyl groups, unreacted hydroxyl groups, and unreacted carboxylic acid groups.

Aspect 11: A composition comprising the light responsive azobenzene functional nanogel of any one of Aspects 1-10, a polymer or a polymer precursor, and a solvent, characterized in that when the composition is exposed to light the light responsive azobenzene nanogel facilitates movement of the polymer or the polymer precursor.

Aspect 12: The composition of Aspect 11 wherein the movement is away from the light.

Aspect 13: The composition of Aspect 11 or 12 wherein the polymer or polymer precursor comprises a dental adhesive.

Aspect 14: The composition of Aspect 13 wherein the dental adhesive comprises one or more radical polymerizable monomers.

Aspect 15: The composition of any one of Aspects 11-14 further comprising a photopolymerization initiator.

Aspect 16: The composition of any one of Aspects 11-15 comprising a free radical initiator.

Aspect 17: The composition of any one of Aspects 11-16 further comprising a catalyst.

Aspect 18: The composition of any one of Aspects 11-17 wherein the solvent comprises water and/or an alcohol, preferably ethanol.

Aspect 19: The composition of any one of Aspects 11-18 wherein the polymer precursor comprises a divinyl monomer, preferably a di(meth)acrylate and a monovinyl monomer, preferably a monoacrylate.

Aspect 20: The composition of Aspect 19 wherein the divinyl monomer comprises one or more of ethylene glycol di(meth)acrylate, hexanediol di(meth)acrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl(meth)acrylate, urethane di(meth)acrylate, 2,2′-bis[4-(3-methacryloxy-2-hydroxy propoxy)-phenyl]propane (bis-GMA), ethoxylated bisphenol-A-di(meth)acrylate and divinyl benzene.

Aspect 21: The composition of Aspect 19 or 20 wherein the monovinyl monomer comprises one or more of C1-C20 alkyl(meth)acrylate, such as ethyl(meth)acrylate or isobornyl(meth)acrylate; an aromatic (meth)acrylate, such as 2-phenoxyethyl(meth)acrylate, benzoyl(meth)acrylate, or phenyl(meth)acrylate; or (meth)acrylic acid.

Aspect 22: The composition of any one of Aspects 11-21 wherein the light responsive azobenzene functional nanogel is present in the composition in amounts of up to 5%, preferably up to 3% by weight based on total weight of the composition.

Aspect 23: The composition of any one of Aspects 11-22 wherein the light responsive azobenzene functional nanogel is present in the composition in amounts of at least 0.2, preferably 0.5, more preferably at least 1% by weight based on total weight of the composition.

Aspect 24: A kit comprising a first container having contents comprising a free radical initiator and a second containing comprising a catalyst wherein the kit includes in at least one of the first container and the second container the light responsive azobenzene functional nanogel of any one of Aspects 1-9 and wherein the kit includes in at least one of the first container and the second container a polymer or a polymer precursor.

Aspect 25: The kit of Aspect 24 wherein the first container includes at least one of (a) the light responsive azobenzene functional nanogel of any one of claims 1-9 and (b) a polymer or a polymer precursor; and the second container includes at least one of (a) the light responsive azobenzene functional nanogel of any one of claims 1-9 and (b) a polymer or a polymer precursor.

Aspect 26: The kit of Aspect 24 or 25 further comprising a mixing mechanism to combine and mix the contents of the first and second containers.

Aspect 27: The kit of any one of Aspects 24-26 wherein the contents of the first container comprise the free radical initiator and the composition of any one of claims 10-20 and the contents of the second container comprise the catalyst and the composition of any one of claims 11-21.

Aspect 28: The kit of any one of Aspects 24-27 wherein the catalyst comprises a peroxide, preferably benzoyl peroxide.

Aspect 29: The kit of any one of Aspects 24-28 wherein the catalyst comprises N, N-dimethyl-p-toluidine.

Aspect 30: A method comprising providing a substrate, applying a composition to the substrate where the composition comprises a photoresponsive azobenzene functional nanogel, a functional component and a solvent, irradiating the composition on the substrate with light to induce movement of the composition on the substrate to a desired location on the substrate.

Aspect 31: The method of Aspect 30 wherein the composition comprises the composition of any one of Aspects 11-23.

Aspect 32: The method of Aspect 30 wherein the composition comprises the polymer precursor further comprising curing to form a polymer.

Aspect 33: The method of claim any one of Aspects 30-32 wherein the step of irradiating causes movement of the composition in a direction away from the light.

Aspect 34: The method of any one of Aspects 30-33 wherein the composition includes a photoinitiator and irradiating initiates the cure.

Aspect 35: The method of Aspect 30 wherein the step of applying comprising mixing the contents of the two containers of the kit of any one of Aspects 24-28 and applying the mixture to the substrate.

Aspect 36: The method of any one of Aspects 30-35 wherein the substrate comprises dentin.

Aspect 37: The method of Aspect 36 wherein the dentin is etched prior to applying the composition.

Aspect 38: The method of any one of Aspects 30-36 wherein the light has a wavelength of 430-480 nm.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). Moreover, stated upper and lower limits can be combined to form ranges (e.g., “at least 1 or at least 2 weight percent” and “up to 10 or 5 weight percent” can be combined as the ranges “1 to 10 weight percent”, or “1 to 5 weight percent” or “2 to 10 weight percent” or “2 to 5 weight percent”).

The disclosure may alternately comprise, consist of, or consist essentially of, any appropriate components herein disclosed. The disclosure may additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

Unless specified to the contrary herein, all test standards are the most recent standard in effect as of the filing date of this application, or, if priority is claimed, the filing date of the earliest priority application in which the test standard appears.

1. A light responsive azobenzene functional nanogel comprising a reaction product of an azobenzene having a first reactive group and a monomer mixture comprising one or more polymerizable monomers having a second reactive group reactive with the first reactive group.

2. The light responsive azobenzene functional nanogel of claim 1 wherein the monomer mixture comprises one or more monovinyl monomers and one or more divinyl monomers and a difunctional chain transfer agent.

3. The light responsive azobenzene functional nanogel of claim 1, wherein the first reactive group of the azobenzene is an ethylenically unsaturated group, a hydroxyl group, or an isocyanate group.

4. The light responsive azobenzene functional nanogel of claim 2
wherein
the divinyl monomer comprises one or more of ethylene glycol di(meth)acrylate, tetraethyleneglycoldi(meth)acrylate (TTEGDMA), urethane dimethacrylate (UDMA), the condensation product of bisphenol A and glycidyl (meth)acrylate, 2,2-bis[4-(3-methacryloxy-2-hydroxy propoxy)-phenyl]propane (bis-GMA), ethoxylated bisphenol-A-di(meth)acrylate (BisEMA), hexanediol di(meth)acrylate, polyethylene glycol dimethacrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl (meth)acrylate, divinyl benzene, bis(meth)acrylamide, or 1,3-diglycerolatediacrylate,
the monovinyl monomer comprises (meth)acrylates and acrylates, styrene and derivatives thereof, vinyl acetate, maleic anhydride, itaconic acid, N-alkyl (aryl) maleimides and N-vinyl pyrrolidone, vinyl pyridine, acrylamide, methacrylamide, N,N-dialkylmethacrylamides or acrylonitrile, and/or
the difunctional chain transfer agent comprises mercaptoethanol, mercaptopropanol, 3-mercapto-2-butanol, 2-mercapto-3-butanol, 3-mercapto-2-methyl-butan-1-ol, 3-mercapto-3-methyl-hexan-1-ol-3-mercaptohexanol, 3-mercaptopropionic acid, or cysteine.

5. The light responsive azobenzene functional nanogel of claim 1, wherein mixture comprises comprising an azobenzene comprises a hydroxyl functional group or an isocyanate functional group and the monomer mixture comprises a polyol, a cyanoalkyl (meth)acrylate, and a diisocyanate.

6. The light responsive azobenzene functional nanogel of claim 1 which is spherically cross-linked and swells in a solvent selected from water, alcohol or combinations there.

7. The light responsive azobenzene functional nanogel of claim 1 comprising one or more of unreacted vinyl groups, unreacted hydroxyl groups, and unreacted carboxylic acid groups.

8. A composition comprising
the light responsive azobenzene functional nanogel of claim 1,
a polymer or a polymer precursor,
and a solvent, characterized in that when the composition is exposed to light the light responsive azobenzene nanogel facilitates movement of the polymer or the polymer precursor.

9. The composition of claim 8 wherein the polymer or polymer precursor comprises a dental adhesive which comprises one or more radical polymerizable monomers and the composition further comprises a photopolymerization initiator.

10. The composition of claim 8 further comprising a catalyst.

11. The composition of claim 8 wherein the polymer precursor comprises a divinyl monomer and a monovinyl monomer.

12. The composition of claim 11 wherein the divinyl monomer comprises one or more of ethylene glycol di(meth)acrylate, hexanediol di(meth)acrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl(meth)acrylate, urethane di(meth)acrylate, 2,2′-bis[4-(3-methacryloxy-2-hydroxy propoxy)-phenyl]propane (bis-GMA), ethoxylated bisphenol-A-di(meth)acrylate and divinyl benzene and the monovinyl monomer comprises one or more of C₁-C₂₀ alkyl(meth)acrylate, such as ethyl(meth)acrylate or isobornyl(meth)acrylate; an aromatic (meth)acrylate, such as 2-phenoxyethyl(meth)acrylate, benzoyl(meth)acrylate, or phenyl(meth)acrylate; or (meth)acrylic acid.

13. The composition of claim 8 wherein the light responsive azobenzene functional nanogel is present in the composition in amounts of at least 0.2 up to 5% by weight based on total weight of the composition.

14. A kit comprising
a first container having contents comprising a free radical initiator and at least one of (a) a light responsive azobenzene functional nanogel comprising a reaction product of an azobenzene having a first reactive group and a monomer mixture comprising one or more polymerizable monomers having a second reactive group reactive with the first reactive group and (b) a polymer or a polymer precursor;
a second container having contents comprising a catalyst and at least one of (a) a light responsive azobenzene functional nanogel comprising a reaction product of an azobenzene having a first reactive group and a monomer mixture comprising one or more polymerizable monomers having a second reactive group reactive with the first reactive group and (b) a polymer or a polymer precursor,
provided that the light responsive azobenzene functional nanogel is present in at least one of the first container and the second container, and the polymer or the polymer precursor is present in at least one of the first container and the second container.

15. The kit of claim 14 further comprising a mixing mechanism to combine and mix the contents of the first and second containers.

16. The kit of claim 14 wherein the contents of the first container comprise the free radical initiator and the light responsive azobenzene functional nanogel, the polymer or the polymer precursor, and a solvent and the contents of the second container comprise the catalyst and the light responsive azobenzene functional nanogel, the polymer or the polymer precursor, and a solvent.

17. The kit of claim 14 wherein the catalyst comprises a peroxide, and/or N,N-dimethyl-p-toluidine.

18. A method comprising
providing a substrate,
applying a composition to the substrate where the composition comprises a photoresponsive azobenzene functional nanogel, a functional component and a solvent,
irradiating the composition on the substrate with light to induce movement of the composition on the substrate to a desired location on the substrate.

19. The method of claim 18 wherein the azobenzene functional nanogel comprises a reaction product of an azobenzene having a first reactive group and a monomer mixture comprising one or more polymerizable monomers having a second reactive group reactive with the first reactive group and the functional component comprises a polymer or a polymer precursor.

20. The method of claim 19 wherein the composition comprises the polymer precursor the method further comprising curing to form a polymer.
